(19) United States
(12) Patent Application Publication
(10) Pub. No.: US 2006/0004089 A1
(43) Pub. Date: Jan. 5, 2006

(54) Use of an l-ascorbic acid salt to prepare a pharmaceutical composition for ophthalmic topical use capable of improving the level of l-ascorbic acid in the eye

(75) Inventors: Mario Pinza, Milano (IT); Lucia Durando, Roma (IT); Leonardo Marchitto, Cupra Marittima (IT); Michele Virno, Roma (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: AZIENDE CHIMICHE RIUNITE , ANGELINI FRANCE, Roma (IT)

(21) Appl. No.: 10/507,025

(22) PCT Filed: Mar. 5, 2003
(86) PCT No.: PCT/EP03/02258

§ 371 Date: Jan. 25, 2005

(30) Foreign Application Priority Data
Mar. 15, 2002 (IT) M12002A000557

(51) Int. Cl. A61K 31/375

(52) U.S. Cl. 514/474

(57) ABSTRACT

Use of a salt of L-ascorbic acid with a pharmaceutically acceptable organic base to prepare a pharmaceutical composition, for ophthalmic topical use, capable of improving the level of L-ascorbic acid in a human eye.

The present invention relates to the use of a salt of L-ascorbic acid with a pharmaceutically acceptable organic base to prepare a pharmaceutical composition, for ophthalmic topical use, capable of improving the level of L-ascorbic acid in the eye. The invention also relates to a therapeutic method that comprises the topical administration of the said salt to an eye whose level of L-ascorbic acid it is desired to improve.

It is known that the levels of L-ascorbic acid in the aqueous humour (1.06 mmol/l; Arshinoff S. A., et al. “Ophthalmology”, chapter 4.20.2, published by Mosby International Ltd., 1999) are about 20 times higher (Brubaker R. F. et al. “Investigative Ophthalmology & Visual Science”, June 2000, vol. 41, No. 7, pp. 1681) than those present in the blood circulation (20-70 μmol/l, Geigy Scientific Tables, vol. 3, page 132, 8th edition 1985, published by Ciba Geigy). In the case of the retina, the levels of L-ascorbic acid in the eye are actually 100 times higher than those present in the blood circulation.

WO 02 01954 discloses the medical use of ascorbic acid and its salts for recovering cellular function following injury. No organic salt of ascorbic acid is specifically mentioned in this document. EP-A-0 781 547 discloses an ophthalmic formulation, for use in eye surgery, comprising sodium hyaluronate, citrate, at least one antioxidant tolerated by the intraocular tissues and a phosphate buffer. The antioxidant may be an ascorbate. More specifically, the ascorbate is sodium ascorbate.

Despite the fact that the role of L-ascorbic acid in the eye has still not been definitively clarified, it is thought that its antioxidant capabilities prevent the crystallization and accumulation of foreign molecules that would interfere with the visual function.

Moreover, it has been proven that:
the level of L-ascorbic acid reduces in an eye suffering from cataracts and glaucoma, and
the systemic administration of L-ascorbic acid reduces the risk of cataracts and decreases the intraocular pressure in an eye suffering from glaucoma.

It is also known that a number of drugs administered ophthalmically cause the undesired effect of reducing the level of L-ascorbic acid in the eye. Typical examples of drugs that cause this undesired effect are steroidal anti-inflammatories such as dexamethasone.

The inventors have now found that the behaviour of salts of L-ascorbic acid with alkali metals is, unexpectedly, different from that of the salts with organic bases, since the cornea is not permeable to the former salts, whereas it is to the latter salts. To the inventors' knowledge, the pronounced difference they found experimentally (Table 1) was not at all foreseeable.

The topical ophthalmic administration of a salt of L-ascorbic acid with a pharmaceutically acceptable organic base is thus advantageous in the prevention and treatment of cataracts and glaucoma. It will moreover be advantageous to combine the administration of the said salt with that of a drug for ophthalmic use that causes a reduction in L-ascorbic acid in the eye, so as to compare the said undesired effect of the said drug.

In a first aspect, the present invention thus relates to the use of a salt of L-ascorbic acid with a pharmaceutically acceptable organic base to prepare a pharmaceutical composition, for ophthalmic topical use, capable of improving the level of L-ascorbic acid in a human eye.

In the course of the present description and in the claims, the term “improve” is used in the relative sense since, when the said salt is administered for therapeutic purposes to an eye presenting a deficiency in ascorbic acid, without this deficiency having been caused by a drug, it effectively has the purpose of increasing the level of L-ascorbic acid in the treated eye. On the other hand, when the said salt is administered to compare the undesired effect of a drug that reduces the level of L-ascorbic acid in the eye, the said salt improves the level of L-ascorbic acid relative to the level that would be reached if the drug was administered alone. Achieving a level intermediate between the higher initial level (basal) and the lower level caused by the administration of a drug having the abovementioned undesired effect thus also forms part of the invention.

Advantageously, the said organic base is chosen from the group comprising tromethamine, N-methylglucosamine, lysine, arginine and omithine.

Tromethamine and lysine are particularly preferred.

The pharmaceutical composition according to the present invention is preferably prepared in suitable dosage forms comprising an effective dose of at least one salt of L-ascorbic acid with a pharmaceutically acceptable organic base and at least one pharmaceutically acceptable inert vehicle.

Examples of suitable dosage forms are creams and sterile solutions. These solutions may be ready-to-use or may be prepared at the time of use by dissolving a sterile powder or lyophilizate in a pharmaceutically acceptable sterile liquid vehicle.

The dosage forms may also contain other conventional ingredients such as: preserving agents, stabilizers, surfactants, dispersants, salts for regulating the osmotic pressure, emulsifiers, colorants and the like.

The amount of the said salt of L-ascorbic acid with a pharmaceutically acceptable organic base in the said pharmaceutically acceptable inert vehicle is typically between 0.1 and 20 mg/ml. Preferably, the said amount is between 0.2 and 10 mg/ml and even more preferably between 0.5 and 2 mg/ml.

The administration of the salt according to the present invention does not involve any undesired effects and may be carried out several times a day. Typically, the said administration is carried out 1 to 24 times a day and preferably from 3 to 12 times a day. However, in the case of compositions also containing another drug, the specific therapeutic regime of the said drug will be followed.

The dosage forms of the pharmaceutical composition of the present invention may be prepared according to techniques, that are well known in pharmaceutical chemistry, including mixing, lyophilization, dissolution, sterilization and the like.

When a salt of L-ascorbic acid with a pharmaceutically acceptable organic base is administered to a patient to compare the reduction in the level of L-ascorbic acid in the eye caused by the administration of another drug, a single dosage form comprising both the said salt and the said drug may advantageously be envisaged.

A typical example of this dosage form is a collyrium comprising an anti-inflammatory drug such as dexamethasone and a salt of L-ascorbic acid with a pharmaceutically acceptable organic base according to the present invention.

When these unit dosage forms, which comprise both a salt of L-ascorbic acid with a pharmaceutically acceptable organic base and a drug capable of reducing the level of L-ascorbic acid in the eye, are in the form of sterile aqueous solutions, the salt according to the present invention may advantageously also act as a buffer.

In a second aspect, the present invention relates to a therapeutic method for improving the level of L-ascorbic acid in a human eye, the said method comprising the topical administration to the said eye of a salt of L-ascorbic acid with a pharmaceutically acceptable organic base.

As mentioned previously, the administration of the salt according to the present invention does not involve any undesired effects and may be carried out several times a day. Typically, the therapeutic method of the present invention comprises from 1 to 24 administrations and preferably from 3 to 12 administrations a day of a pharmaceutical form comprising from 0.1 to 20 mg/ml of the said salt of L-ascorbic acid with a pharmaceutically acceptable organic base. Preferably, the said pharmaceutical form comprises from 0.2 to 10 mg/ml and even more preferably from 0.5 to 2 mg/ml of the said salt of L-ascorbic acid with a pharmaceutically acceptable organic base.

However, when the salt according to the present invention is administered in combination with another drug, the specific therapeutic regime of the said drug will be followed.

The following examples serve to illustrate the invention without, however, limiting it.

EXPERIMENTAL SECTION

| Composition A | | |
|---|---|---|
| Lysine ascorbate | 0.909 | g |
| Natrosol 250 | 1.200 | g |
| NaCl | 0.350 | g |
| Distilled water | qs 100 | ml |
| pH | 5.3-5.6 | |
| Composition B | | |
| Tromethamine ascorbate | 0.838 | g |
| Natrosol 250 | 1.200 | g |
| Distilled water | qs 100 | ml |
| pH | 5.0-5.6 | |

### Comparative Composition 1

The collyrium Luxazone™ sold in Italy by the company Allergan was used. Said collyrium consists of an aqueous 0.2% solution. The other ingredients are monosodium phosphate, sodium sulphite heptahydrate, sodium chloride, benzalkonium chloride, hydroxypropylcellulose and polysorbate 80.

### TEST 1

| Comparative composition 2 | | |
|---|---|---|
| Sodium ascorbate | 0.562 | g |
| Natrosol 250 | 1.200 | g |
| KH₂PO₄ | 1.000 | g |
| K₂HPO₄ | 0.270 | g |
| NaCl | 0.250 | g |
| Distilled water | qs 100 | ml |
| pH | 5.0-5.5 | |

### Transcorneal Passage of L-ascorbic Acid

1. Aim of the Experiment

The purpose of the test was to confirm the passage of L-ascorbic acid across the cornea under experimental conditions of a reduced level of endogenous L-ascorbic acid in the aqueous humour.

The experimental model used involved measuring the level of L-ascorbic acid in the aqueous humour first under basal conditions and then after having induced a reduction in the level of endogenous L-ascorbic acid in the aqueous humour by means of chronic treatment with Comparative Composition 1 of the prior art.

The effect on the level of endogenous L-ascorbic acid of Comparative Composition 2 and of Compositions A and B according to the invention was also evaluated in the same way.

2. Experimental Model

Male New Zealand White rabbits (2.5-4 kg) were used.

The animals, divided into groups of 3 animals each, were treated according to the following scheme:

| | |
|---|---|
| Group A1 | :Comparative Composition 1 |
| Group A2 | :Comparative Composition 1 + |
| | Comparative Composition 2 |
| Group B1 | :Comparative Composition 1 |
| Group B2 | :Comparative Composition 1 + |
| | Composition A of the invention |
| Group C1 | :Comparative Composition 1 |
| Group C2 | :Comparative Composition 1 + |
| | Composition B of the invention |

The animals were treated topically (100 μl/eye) in both eyes 3 times a day for about 2 weeks.

The measurement of the level of endogenous L-ascorbic acid in the aqueous humour was carried out before the start of the treatment (basal value) and 2 weeks after the start of the treatment (14th day).

The level of endogenous L-ascorbic acid was measured in aqueous humour removed by paracentesis, under general and topical anaesthesia, from the right and left eye alternately.

The determination of the L-ascorbic acid was carried out using a Merck RQflex® plus reflectometer, working according to the Merck test 1.16981.0001 which is based on the property of ascorbic acid to reduce yellow molybdophosphoric acid to blue phosphomolybdenum and reflectometric measurement of the latter.

3. Results

The levels of L-ascorbic acid (mg/l) in the aqueous humour of rabbits measured on day 0 and on day 14 are shown in the following table, in which the value in parentheses indicates the change relative to the basal reading.

TABLE I

| Group | Day 0 | Day 14 |
|---|---|---|
| A1 | 211 | 68 (−68%) |
| A2 | 197 | 63 (−68%) |
| B1 | 192 | 49 (−74%) |
| B2 | 198 | 78 (−61%) |
| C1 | 149 | 69 (−53%) |
| C2 | 162 | 101 (−38%) |

The abovementioned results show that:
in Group A1, a pronounced reduction (68%) in the level of endogenous L-ascorbic acid was measured after 14 days of treatment with dexamethasone alone (Comparative Composition 1);
in Group A2, this reduction was not affected by simultaneous administration of a salt of ascorbic acid with a mineral base (sodium ascorbate: Comparative Composition 2);
in Group B1, the reduction in the level of endogenous L-ascorbic acid after 14 days of treatment with dexamethasone alone (Comparative Composition 1) was 74%;
in Group B2, this reduction was affected by simultaneous administration of a salt of ascorbic acid with an organic base (lysine ascorbate: Composition A). Specifically, in this case, the reduction in the level of endogenous L-ascorbic acid after 14 days of treatment was 61% rather than 74%;
in Group C1, the reduction in the level of endogenous L-ascorbic acid after 14 days of treatment with dexamethasone alone (Comparative Composition 1) was 53%;
in Group C2, this reduction was effectively affected by with the simultaneous administration of a salt of ascorbic acid with an organic base (tromethamine ascorbate: Composition B). Specifically, the reduction in the level of endogenous L-ascorbic acid after 14 days of treatment was only 38%, rather than 53%.

The abovementioned results therefore prove that, unlike the salts with mineral bases, the salts of ascorbic acid with organic bases pass through the cornea. This effect was particularly pronounced in the case of the tromethamine salt.

1. A composition comprising a salt of L-ascorbic acid and a pharmaceutically acceptable organic base.

2. The composition according to claim 1, wherein organic base is selected from the group consisting of tromethamine, N-methylglucosamine, lysine, arginine and omithine.

3. The composition according to claim 1, wherein the organic base is tromethamine or lysine.

4. The composition according to claim 1, wherein the composition is in the form of a cream or a sterile solution.

5. The composition according to claim 1, wherein the said composition contains from 0.1 to 20 mg/ml of the salt of L-ascorbic acid with the pharmaceutically acceptable organic base, and further comprises at least one pharmaceutically acceptable inert vehicle.

6. The composition according to claim 5, wherein the composition contains from 0.2 to 10 mg/ml of the salt of L-ascorbic acid with the pharmaceutically acceptable organic base.

7. The composition according to claim 5, wherein the composition contains from 0.5 to 2 mg/ml of the salt of L-ascorbic acid with a the pharmaceutically acceptable organic base.

8. The composition according to claim 1, wherein the composition is in the form of a sterile collyrium comprising the salt of L-ascorbic acid with lysine or with tromethamine.

9. The composition according to claim 8, wherein the composition further comprises an anti-inflammatory drug.

10. The composition according to claim 9, wherein the anti-inflammatory drug is dexamethasone.

11. A therapeutic method comprising topically administering a composition, comprising an effective amount of a salt of L-ascorbic acid with a pharmaceutically acceptable organic base, to an eye of a subject in need thereof.

12. The method according to claim 11, wherein the organic base is selected from the group consisting of tromethamine, N-methylglucosamine, lysine, arginine and ornithine.

13. The method according to claim 11, wherein the organic base is tromethamine or lysine.

14. The method according to claim 11, wherein the composition is administered 1 to 24 times a day in the form of a sterile pharmaceutical dosage containing from 0.1 to 20 mg/ml of the salt.

15. The method according to claim 11, wherein the composition is administered 3 to 12 times a day in the form of a sterile pharmaceutical dosage containing from 0.1 to 20 mg/ml of the salt.

16. The method according to claim 11, wherein the composition is administered 1 to 24 times a day in the form of a sterile pharmaceutical dosage containing from 0.2 to 10 mg/ml of the salt.

17. The method according to claim 11, wherein the composition is administered 3 to 12 times a day in the form of a sterile pharmaceutical dosage containing from 0.2 to 10 mg/ml of the salt.

18. The method according to claim 11, wherein the composition is administered 1 to 24 times a day in the form of a sterile pharmaceutical dosage containing from 0.5 to 2 mg/ml of the salt.

19. The method according to claim 11, wherein the composition is administered 3 to 12 times a day in the form of a sterile pharmaceutical dosage containing from 0.5 to 2 mg/ml of the salt.

20. The method according to claim 11, wherein the composition further comprises an anti-inflammatory drug.

21. The method according to claim 20, wherein the anti-inflammatory drug is dexamethasone.
